(19) United States
(12) Patent Application Publication
(10) Pub. No.: US 2021/0030477 A1
(43) Pub. Date: Feb. 4, 2021

(54) COMPUTER ASSISTED IMPLANT PLACEMENT

(71) Applicant: THINK SURGICAL, INC., Fremont, CA (US)

(72) Inventors: Joel Zuhars, Fremont, CA (US); Daniel Patrick Bonny, Fremont, CA (US)

(73) Assignee: THINK SURGICAL, INC., Fremont, CA (US)

(21) Appl. No.: 17/074,981

(22) Filed: Oct. 20, 2020

Related U.S. Application Data
(63) Continuation-in-part of application No. 15/542,707, filed on Jul. 11, 2017, now abandoned.
(60) Provisional application No. 62/104,657, filed on Jan. 16, 2015.

(51) Int. Cl. A61B 34/10; A61B 34/20; A61B 34/30; B25J 15/00

(52) U.S. Cl. CPC A61B 34/10; A61B 34/20; A61B 2034/2059; B25J 15/0019; A61B 2034/108; A61B 34/30

(57) ABSTRACT

A method for implantation of non-spherical or asymmetric implants is provided that includes devising a pre-surgical plan with pre-operative planning software operating on a computer to define at least one shape, orientation, type, size, geometry, or placement of an implant having at least a head component or a cup component in, on, or relative to an operative bone of a subject, the head component or the cup having at least two cross-sectional radii of different dimensions in, on, or relative to an operative bone of a subject. A computer-assisted surgical device is used to place the implant. The implant is positioned in, on, or relative to the bone by the computer-assisted surgical device in accordance with the pre-surgical plan. A system for devising a pre-surgical plan for the implant, and positioning the implant in, on, or relative to a bone in accordance with the pre-surgical plan is also provided.

### RELATED APPLICATIONS

This continuation-in-part application claims priority benefit of U.S. Nonprovisional application Ser. No. 15/542,707 filed Jul. 11, 2017 which is a US National Phase Application of PCT/US2016/03533 filed Jan. 15, 2016, which in turn claims priority benefit of U.S. Provisional Application Ser. No. 62/104,657 filed Jan. 16, 2015; the contents of which are hereby incorporated by reference.

### FIELD OF THE INVENTION

The present invention relates generally to computer-assisted orthopedic surgery, and more specifically to computer-assisted placement of non-spherical or asymmetric implants that require a precise configuration.

### BACKGROUND OF THE INVENTION

In total hip arthroplasty, implants are used to replace the ball and socket joint of the hip to restore a subject's natural function. The joint is exposed and the femur and acetabulum are prepared, using reamers and broaches, to receive the implants. The femoral implant generally consists of a stem, neck, and ball portion while the acetabular implant generally consists of an outer and inner shell. FIG. 1 is a prior art perspective view of a modular type femoral hip implant 100 that is designed with the stem 105, neck 103, and head 101 as separate components. Manufacturers provide different sizes and shapes for each component of a modular type femoral implant so the components may be assembled by the surgeon in a configuration that best fits the subject. While there are many different types of hip implants, one universal design characteristic is the rotational symmetry of the modular head component 101 about the longitudinal implant neck axis 107.

However, research has shown that the true shape of a healthy human femoral head is an ellipsoid rather than a sphere. There have been many proposed clinical benefits to the elliptical shape including improved mechanical properties and cartilage health. For example, a group performed computer simulated studies on the mechanical behavior of elliptical shaped heads vs. spherical shaped heads. The study reported that the ellipsoid model behaves better than a sphere in terms of acetabular deformation and acetabular peak stresses under static conditions.

Similarly, the components of an acetabular implant are generally designed as perfect hemispheres. However, the acetabulum in fact has a morphologically undulating rim. The undulations consist of peaks and valleys where important muscles and tendons naturally align and follow. For example, the psoas valley on the anterior rim of the acetabulum provides an anatomical path for the iliopsoas tendon. It has been shown that protruding or mal-aligned acetabular components may cause iliopsoas impingement, which may lead to tendon irritation and tearing. As the manufacturing of asymmetric, non-spherical and/or implants with unique features are possible, the placement of the components within the subject requires a high degree of precision and accuracy.

Recently, with the advancements in three dimensional (3-D) printing, subject specific implants have been proposed as a possible alternative to traditional implants. 3D printed implants may take advantage of the natural and healthy shaped anatomy that provides a clinical benefit to the subject and to the longevity of the implant. However, there is still a need to optimally place the implants in the subject intra-operatively to ensure the orientation and any crucial landmarks of the implant are in the correct location.

Thus, there exists a need to provide the surgeon and subject with an implant that takes advantage of the natural shapes of the anatomy to provide a better clinical outcome. There further exists a need for the precise placement of the implant in the subject.

### SUMMARY OF THE INVENTION

A method for implantation of non-spherical or asymmetric implants is provided that includes devising a pre-surgical plan with pre-operative planning software operating on a computer to define at least one shape, orientation, type, size, geometry, or placement of an implant having at least a head component or a cup component in, on, or relative to an operative bone of a subject, the head component or the cup having at least two cross-sectional radii of different dimensions. A computer-assisted surgical device is used to place the non-spherical or asymmetric implant. The implant is positioned in, on, or relative to the bone by the computer-assisted surgical device in accordance with the pre-surgical plan.

A system for devising a pre-surgical plan for a non-spherical or asymmetric implant and implanting the non-spherical or asymmetric implant on, in, or relative to a bone in accordance with the pre-surgical plan is also provided.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 depicts a typical modular hip implant of the prior art with a femoral head rotationally symmetric about the implant neck axis in accordance with embodiments of the invention;

FIG. 2 depicts an inventive modular femoral head implant design that has only two orders of rotational symmetry along three axes in accordance with embodiments of the invention;

FIG. 3 depicts a modular assembly of the inventive head design of the implant and the effect of rotating the component about the neck axis in accordance with embodiments of the invention;

FIGS. 4A and 4B illustrates a femoral hip implant having an non-spherical head, a neck, and a stem in accordance with embodiments of the invention, where FIG. 4A illustrates a unitary or monolithic femoral hip implant, and FIG. 4B illustrates a unitary or monolithic stem and neck with a modular head.

FIG. 5 illustrates an acetabular cup implant with unique features in accordance with embodiments of the invention;

FIG. 6 depicts a modular neck rotating about the stem to an optimal orientation in accordance with embodiments of the invention;

FIG. 7 illustrates a method to fix the femoral head on the neck in a preferred orientation in accordance with embodiments of the invention;

FIG. 8 illustrates a femoral hip implant and acetabular cup implant positioned in the femur and acetabulum, respectively, using computer assistance in accordance with embodiments of the invention;

FIG. 9 illustrates a computer-assisted surgical system including a surgical robot and a pre-operative planning workstation in accordance with embodiments of the invention;

FIG. 10 illustrates an end-effector of a surgical robot assembled to an implant for placing the implant in, on, or relative to a bone in accordance with embodiments of the invention; and

FIG. 11 illustrated a tracked device assembled to an implant to assist in positioning the implant in, on, or relative to a bone in accordance with embodiments of the invention.

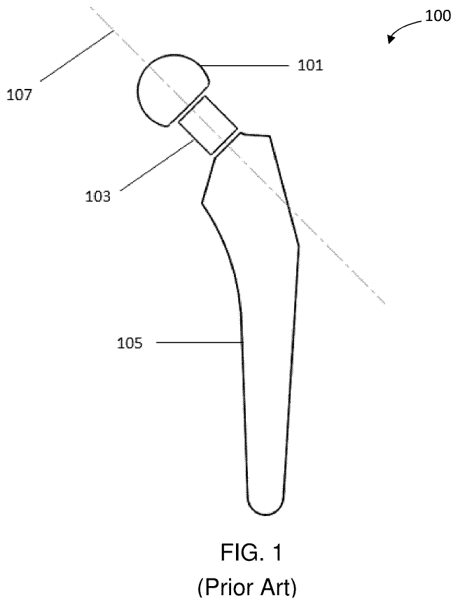

### DESCRIPTION OF THE INVENTION

The invention disclosed herein describes asymmetrical, non-spherical, and/or implants with unique features and methods for implantation, but more particularly to the planning and execution of joint replacement surgery with asymmetrical, non-spherical, and/or implants with unique features with computer-assisted devices.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

The invention disclosed herein has utility for the implantation of non-spherical, asymmetric, and/or implants with unique features that provide a clinical benefit to the subject and/or the longevity of the implant. It should be appreciated that research suggests that naturally shaped implants may provide a clinical benefit especially if precisely placed in the proper location and orientation in the subject.

Reference will be made herein to the replacement of hip joints and knee joints and it should be understood that the present invention may be applied to other joints within the body and any other bones found within the body. These other joints that are repaired through resort to the present invention illustratively include the hip joint, shoulder joint, ankle joint, wrist joint, finger joint, toe joint, or other joint. As used herein, a subject is defined as a human; or an animal of a non-human primate, a horse, a cow, a sheep, a goat, a dog, a cat, a rodent and a bird; and a non-living cadaver of any of the aforementioned.

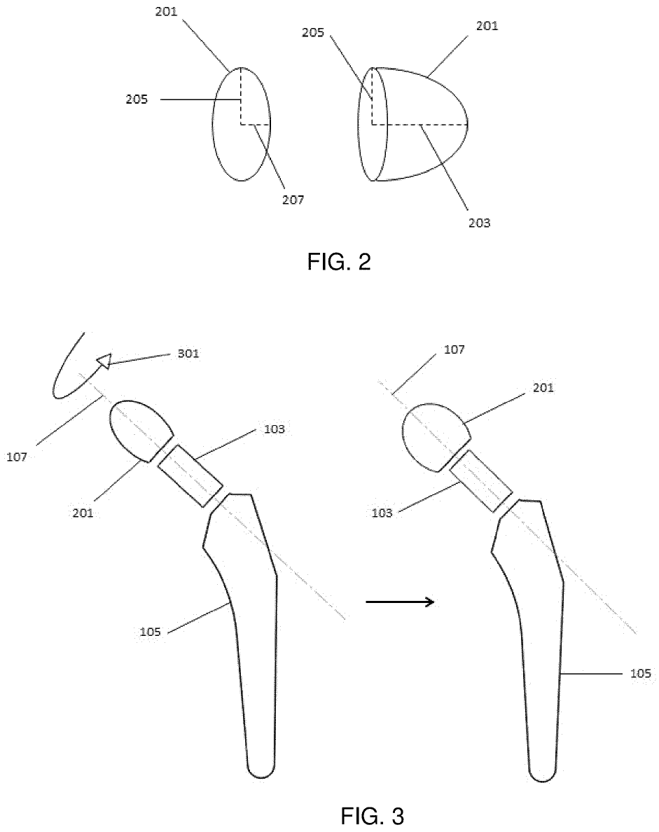

Referring now to the figures, FIG. 2 depicts a non-spherical modular femoral head implant 201 that has only two orders of rotational symmetry about axis 203 in accordance with embodiments of the invention. The femoral head implant 201 may have different dimensions about all three axes 203, 205 and 207. In a specific embodiment, the head implant 201 is designed as an ellipsoid. The radius along axis 203 may be greater than the radius along axis 205. The radius along axis 205 is greater than the radius along axis 207. The non-spherical modular femoral head implant 201 therefore has a first cross-sectional radius (i.e., axis 205) greater than a second cross-sectional radius (i.e., axis 207). In a specific embodiment, the rotational axis 203 of the femoral head implant 201 aligns with neck axis 107. With respect to FIG. 3, when the head implant 201 is rotated (represented by arrow 301) about the neck axis 107, the geometric relationship of the femoral head implant 201 changes with respect to the neck 103 and stem 105. In traditional total hip arthroplasty, manually placing a non-spherical femoral head implant 201 on the neck 103 in the correct orientation to best match a pre-surgical plan or the true shape of the subject's anatomy would be difficult. However, this limitation may be overcome with the use of a computer-assisted surgical device as described below. Axial alignment of axis 107 relative a complementary component (e.g., a cup implant (401, 410), or neck component 103) in a desired orientation is now possible with resort to computer-assisted surgical device positioning. As a result, complications of conventional surgeries, such as impingement, are inhibited.

In specific embodiments, any combination of two or more differing radii between two or more axes may be accomplished whereby FIG. 2 simply illustrates one example of an implant with different dimensions about three axes 203, 205, and 207. Other implants or implant components may likewise be asymmetric, non-spherical, or have unique features that would provide a clinical benefit to the subject. For example, an acetabular cup implant may likewise be non-spherical and in the shape of an ovoid or ellipsoid to receive a non-spherical femoral head 201. An implant with unique features may also benefit from placement with a computer-assisted surgical device. For example, an acetabular cup implant 401 with unique features is illustratively shown in FIG. 5. In one embodiment, the acetabular cup implant 401 has an extruding portion 403 at the top of the implant. The extruding portion may allow for more stability, congruency, and load transfer for use with a traditional femoral head implant or a femoral head implant that is asymmetric, non-spherical, and/or has a unique feature. In another embodiment, the acetabular cup implant 401 may have a recess 405 at the rim that may provide additional space for the iliopsoas tendon as an example. Due to the unique features, manual implantation may prove difficult to get the unique features in the correct position and/or orientation. Therefore, a computer-assisted device may be used to precisely place the implant so the unique features are in an optimal position to provide the best clinical benefit to the subject.

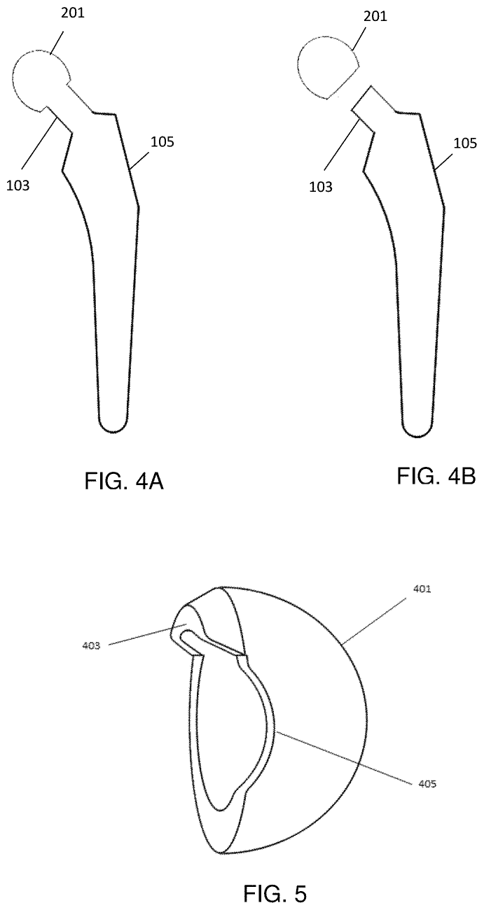

With reference to FIGS. 4A and 4B, a femoral hip implant having a non-spherical head implant 201 is shown. FIG. 4A illustrates a unitary or monolithic femoral hip implant where the stem 105, neck 103, and head 201 are a single unit. FIG. 4B illustrates a femoral hip implant having a unitary or monolithic stem 105 and neck 103, and a modular head 201. The neck 103 and stem 105 may be referred to herein as complementary implant components, which may also describe implant liners, trays, or other components that interact with the implant. Computer-assistance may be used to position the unitary femoral hip implant of FIG. 4A, and position or orient a modular non-spherical head 201 relative to the neck 103 and stem 105 of the femoral hip implant shown in FIG. 4B.

As shown above total hip arthroplasty is one implementation that benefits from embodiments the invention, and the use of asymmetric, non-spherical and/or implants with unique features may be advantageous for other applications as well. For example, the implants and computer-assisted implantation may be used in other surgical contextual locations such as the knee joint, hip joint, spine, shoulder joint, elbow joint, ankle joint, jaw, tumor site, joints of the hand or foot, and other appropriate surgical sites. In a specific embodiment, a hip resurfacing implant may have an elliptical shape (e.g., the femoral head implant 201 may be positioned directly on a resurfaced femoral head typical of hip resurfacing procedures).

### Pre-Operative Implant Planning

In a specific inventive embodiment, pre-operative planning software may be used to determine the best shape, orientation, type, size, geometry, placement, or a combination of any of the aforementioned of an implant in, on, or relative to the operative bone. The operative bone may be represented in the pre-operative software as two-dimensional images or three-dimensional virtual models as known in the art. The pre-operative planning software may have a database of pre-loaded manufacturer implants that the user may choose from to optimally plan the surgery. The manufacturer implants may include implants with non-spherical, asymmetric, or unique features that are regulatory approval. In another specific embodiment, generic virtual models of an implant may be chosen by the user, where the shape of the generic virtual model may be modified and then sent to a third party to be manufactured. For example, the generic virtual model of a femoral head may be a sphere, represented as a triangular mesh, where the user may adjust the diameters or radii into an elliptical shape that the user deems is the most appropriate for the subject. In another embodiment, the pre-operative planning software creates a virtual model of the bone and automatically creates a subject specific implant according to the subject's anatomy. The shape or geometry of the subject specific implant may be created based on the natural and healthy shape of the bone in cases of bone deformity. In certain embodiments, the natural or healthy shape of the subject's contralateral side may be used to create the subject specific implant.

In a specific embodiment, the implant components are modular. The pre-operative planning software allows the user to select or design individual components of the overall implant. For example, the user may choose from a database of different stems, necks, and femoral head implants that may be assembled virtually that provide the best clinical outcome and/or implant survival rate. In one embodiment, the pre-operative planning software allows the user to choose from one or more manufactured modular components while allowing the customization of any of the remaining components. For example, the user may choose a regularly manufactured stem and neck while the femoral head implant is custom designed. In a specific embodiment, the pre-operative planning software may automatically ensure that the custom component is designed to precisely fit the regularly manufactured components. For example, if the femoral head is automatically designed by the pre-operative planning software or by the user then the software will ensure the head may be optimally assembled on the desired neck component.

In another specific embodiment, when an implant is non-spherical, asymmetric or has a unique feature, the pre-operative planning software may put constraints on, and/or automatically assist in the choice and/or design of the opposing component(s) to ensure optimal fit and performance. For example, the user may design or choose an elliptical femoral head implant first where the shape of the femoral head implant puts constraints on the design or reduces the number of choices for the acetabular component. Therefore, the software ensures all of the components of the implant procedure may be optimally and safely assembled within the subject and according to the user's pre-operative plan.

### Computer-Assisted Implant Placement

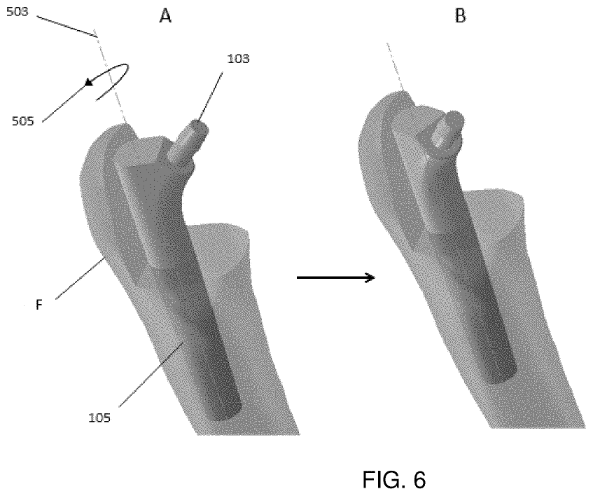

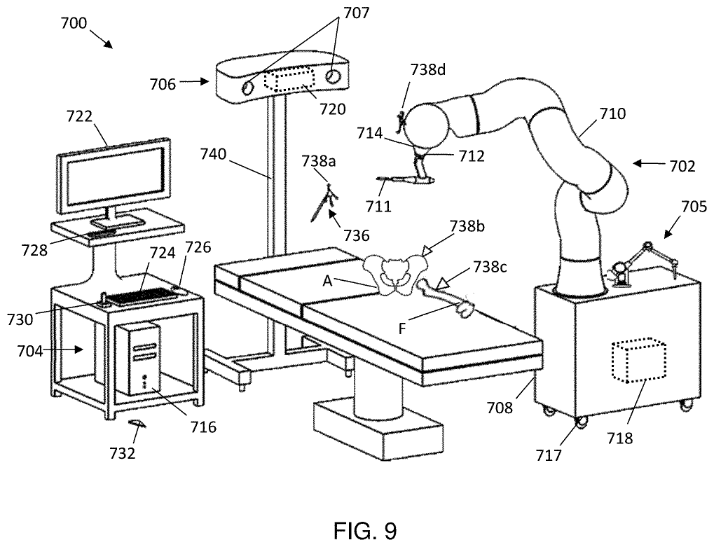

Intra-operatively, a computer-assisted surgical device may assist a surgeon in preparing the bone and precisely placing the non-spherical, asymmetric, and/or implant with a unique feature. Examples of computer-assisted surgical devices include a serial-chain manipulator system as described with reference to FIG. 9, a parallel robotic system, a haptically controlled robotic system, or a hand-held robotic system, such as those described in U.S. Pat. Nos. 5,086,401, 7,206,626, and 8,961,536 all of which are hereby incorporated by reference in their entirety. The computer-assisted surgical device may also refer to a navigated or tracked set of surgical instruments, such as an optically tracked digitizer, or a tracked device that may assemble to a portion of the implant to determine the position and orientation of the implant in, on, or relative to the bone. In a specific inventive embodiment, the computer-assisted surgical device precisely prepares the femoral cavity according to the pre-operative plan so the desired alignment, fit, and fill of the stem component is achieved. In certain cases, the modular neck component may fit on the stem in different orientations. With respect to FIG. 6, a modular neck 103 is shown that may be fixed into place onto the stem 105 within the femur ‘F’ at different orientations. In one embodiment, the computer-assisted surgical device knows the position and orientation of the femur using known registration techniques such as point to surface registration as described in U.S. Pat. No. 6,033,415, which is hereby incorporated by reference in its entirety. The device also knows the orientation of the milled cavity from the cut instructions created during the pre-operative plan. The device may then optimally place the modular neck component 103 in the proper orientation with respect to the femur. For instance, as shown in FIG. 5, the device may rotate (shown by arrow 505) the modular neck 103 to the desired anteversion defined in the pre-surgical plan (from A to B) about axis 503. The neck 103 may then be fixed into place manually by the surgeon or by the surgical device using a fixation technique known in the art such as screws, press-fit, or pins. The device may also rotate recess 405 of the acetabular component 401 to obtain a desired acetabular anteversion or a combined femoral and cup anteversion.

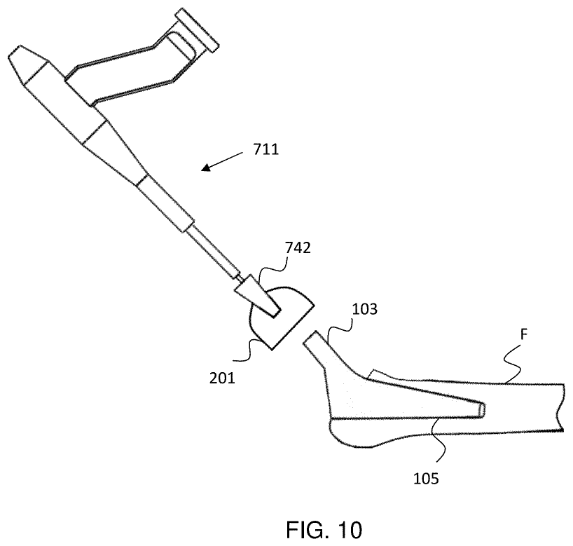

In another specific embodiment, with respect to FIG. 2, the computer-assisted surgical device may optimally orient a modular femoral head implant 201 on the neck 103. The device may assemble and/or attach to the head 201 using a technique known in the art such as a gripping clamp, a magnet, a reference hole on the implant that receives the end effector of the device (as shown in FIG. 10). In one embodiment, a small receiving portion, such as a hole, is located in a specific location on the implant that provides the surgical device with the orientation of the implant relative to the end effector. In another embodiment, a digitizer may be used to collect points on the implant that may be used for registration with a virtual model of the implant to provide the device with the orientation of the implant. Once the initial orientation of the implant is known, the device may precisely orient the implant in the desired orientation. For example, the implants may be registered by digitizing a set of points on the implant so the device knows the initial orientation of the modular head implant 201 and the orientation of the neck 103, and subsequently rotate (shown by arrow 301) the head implant 201 to the desired orientation. In some inventive embodiments, the computer-assisted surgical device also translates the correctly oriented modular head implant 201 into contact with the neck 103. It will be appreciated, that since an ellipsoidal femoral head has a major axis 205 and a minor axis 207, rotation of the femoral head 201 about the neck 103 may affect the femoral anteversion and/or the combined femoral and cup anteversion even by a slight amount (e.g., 0.01 degrees). Therefore, in particular embodiments, rotation of the femoral head implant 201 about the neck 103 with the computer-assisted surgical device to a desired orientation may also be performed to achieve a desired femoral or combined anteversion.

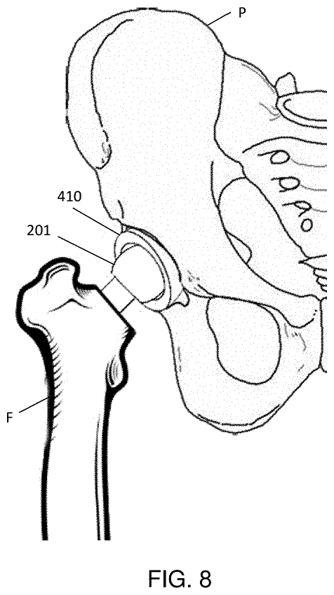

In a particular embodiment, with reference to FIG. 8, a computer-assisted surgical device may be used to position one or more implants in one or more bones during total hip arthroplasty. The femur ‘F’ and/or acetabulum of the pelvis T′ may be prepared using computer-assistance (e.g., milled with a surgical robot), or prepared using conventional manual techniques. The computer-assisted surgical device may then assist in positioning, orienting, or aligning the implants in, on, or relative to the prepared femur ‘F’ and the prepared acetabulum, or relative to a complementary implant components. In one example, the surgeon may install the stem 105 in the femoral cavity and assemble the neck 103 to the stem 105. The head implant 201 is then assembled to the computer-assisted surgical device, which then places the head implant 201 on the neck 103, and rotates the head implant 201 about the neck 103 to the desired orientation as defined in the pre-surgical plan. For the prepared acetabulum, a cup implant 410 having two cross-sectional radii of different dimensions is assembled to the computer-assisted surgical device. The computer-assisted surgical device then places the cup implant 410 in the acetabulum and rotates the cup 410 to the desired orientation as defined in the pre-surgical plan. After the cup 410 is installed, a cup liner may be assembled to the cup implant 410. In this instance, positioning the implants using computer-assistance is particularly advantageous to ensure the head 201 rotates and moves within the cup 410 as prescribed.

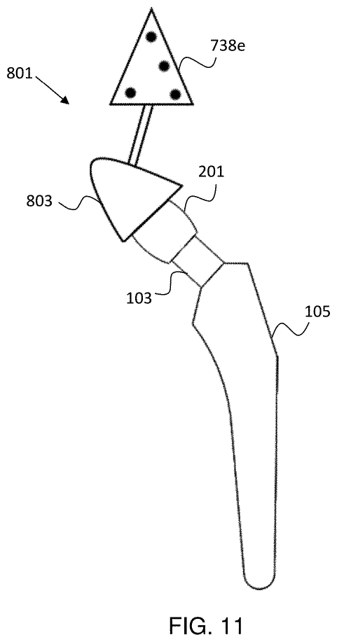

In a specific embodiment, with reference to FIG. 11, a computer-assisted surgical device includes a navigated or tracked device 801 to determine the position and/or orientation of the non-spherical, asymmetric, and/or unique feature implant in, on, or relative to the bone. The tracked device 801 may include an assembly member 803 and a tracking array 738e. The assembly member 803 makes contact or connects with a portion of the implant 201, while the tracking array 738e permits a tracking system to track the position and orientation of the tracked device 801. In particular embodiments, the tracked device 801 may only contact or connect with the implant 201 in a unique orientation such that the position and/or orientation of the implant 201 is known by a tracking system and/or surgical system when the assembly member 803 assembles with the implant 201. For example, the assembly member 803 may have an ellipsoidal cup that matches the shape of an ellipsoidal femoral head implant such that the assembly member 803 only fits on the implant 201 in two orientations. An example of using the tracked device 801 may include the following steps. After pre-operative planning and the generation of a pre-surgical plan, a user or computer-assisted device prepares the femur to receive the stem 105 and neck 103 in the planned position and orientation. The stem 105 and neck 103 are implanted into the prepared cavity. A user may then place the femoral head implant 201 on the neck 103 in any orientation. The user then assembles the tracked device 801 to the femoral head implant 201. The tracking system and/or surgical system then provides the user, via a graphical user interface (GUI), with the initial orientation of the femoral head implant 201 relative to the bone or the other implant components. The GUI may further provide the user with instructions on how to adjust (e.g., rotate) the femoral head implant 201 to best match the pre-surgical plan. The user may then adjust the orientation of the femoral head implant 201 on the neck 103 with real-time feedback from the system, until the orientation of the femoral head implant 201 matches that of the pre-surgical plan. Once in the correct orientation, the femoral head implant 201 is fixed in place on the neck 103 either manually or with resort to the computer-assisted surgical device. It should be appreciated that the same may be applied to any non-spherical, asymmetric, or unique feature implant as described herein.

Component Fixation

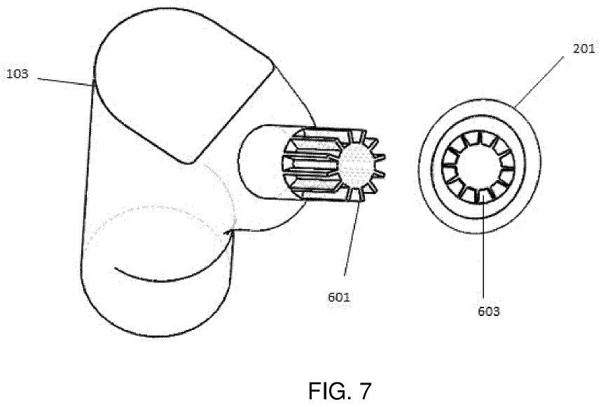

The asymmetrical, non-spherical, and/or implant with a unique feature may be designed to be fixed in the desired orientation relative to the other implant components and/or relative to the bone. In a specific embodiment, with respect to FIG. 7, the modular neck 103 that receives the femoral head implant 201 may contain teeth and grooves 601 that allow the head 201 to be fixed when placed in the desired orientation on the neck 103. The portion of the femoral head implant 201 the fits over the neck 103 may contain corresponding teeth and grooves 603 that tightly fix the implant 201 into the desired orientation. Half the number of grooves would correspond to the number of different orientations the implant may be fixed for an implant with an axis of rotational symmetry having an order of 2. Depending on the number and design of the grooves, multiple orientations may exist that require a surgical device to reference the neck 103 and the femoral head 201 in order to optimally place the femoral head 201. In a specific embodiment, when only a few orientations exist for the femoral head 201 to be placed on the neck 103, the user may directly place the head 201 and fix the head 201 into place. A device or pre-operative plan may be used to help assist the surgeon in placing the component head 201. For example, the teeth and grooves may be numbered whereby after surgical planning the surgeon knows that groove ‘x’ on the neck should fit with tooth ‘y’ of the head.

In another specific inventive embodiment, the implant may be fixed in the desired orientation using an interference fit and/or biocompatible reagents such as Poly(methyl methacrylate) (PMMA). The computer-assisted device, upon registering the implant, may optimally place the correct amount of the reagent at specific locations on the implant that are known to provide a sufficient fix. The computer-assisted device may then place the implant in the correct orientation. In specific embodiments, the implants may be made of materials whereby upon contact with a reagent causes a biocompatible reaction that fixates the two contacting surfaces. In a specific embodiment, during the pre-planning stage, once the location and orientation of all the components have been placed, the components may be selected, designed and/or tailored so that the connecting portions may be designed to fix only in the desired orientations.

Robotic Surgical System

FIG. 9 depicts a computer-assisted surgical system 700 in the context of an operating room (OR) to prepare a femoral bone ‘F’ and acetabulum ‘A’ during total hip arthroplasty and implanting a non-spherical or asymmetric implant, or an implant having a unique feature as previously described. The surgical system 700 includes a surgical robot 702 (or manipulator system), a computing system 704, and an optional tracking system 706. The surgical robot 702 may include a movable base 708, a manipulator arm 710 connected to the base 708, an end-effector 711 located at a distal end 712 of the manipulator arm 710, and a force sensor 714 positioned proximal to the end-effector 711 for sensing forces experienced on the end-effector 711. The base 708 includes a set of wheels 717 to maneuver the base 708, which may be fixed into position using a braking mechanism such as a hydraulic brake. The base 708 may further include an actuator to adjust the height of the manipulator arm 710. The manipulator arm 710 includes various joints, links, and sensors (e.g., encoders) to accurately manipulate the end-effector 711 in various degrees of freedom. The joints are illustratively prismatic, revolute, spherical, or a combination thereof. The end-effector 711 may be motor-driven end-mill, cutter, drill-bit, or other bone removal device.

The computing system 704 may generally include a planning computer 716; a device computer 718; a tracking computer 720; and peripheral devices. The planning computer 716, device computer 718, and tracking computer 720 may be separate entities, one-in-the-same, or combinations thereof depending on the surgical system. Further, in some embodiments, a combination of the planning computer 716, the device computer 718, and/or tracking computer 720 are connected via wired or wireless communication. The peripheral devices allow a user to interface with the surgical system components and may include: one or more user-interfaces, such as a display or monitor 722 to display a graphical user interface (GUI); and user-input mechanisms, such as a keyboard 724, mouse 726, pendent 728, joystick 730, foot pedal 732, voice control software (not shown), or the monitor 722 that in some inventive embodiments has touchscreen capabilities.

The planning computer 716 contains hardware (e.g., processors, controllers, and/or memory), software, data and utilities that are in some inventive embodiments dedicated to the planning of a surgical procedure, either pre-operatively or intra-operatively. This may include: reading pre-operative bone data; displaying pre-operative bone data; manipulating pre-operative bone images (e.g., image segmentation); constructing three-dimensional (3D) virtual models; storing computer-aided design (CAD) files; designing custom shaped implants; planning the position and/or orientation (POSE) of implants in, on, or relative to one or more bones or modular implant components; providing various functions or widgets to aid a user in planning the surgical procedure; and generating surgical plan data. The final surgical plan may include: pre-operative bone data; patient data; registration data including the position of a set of points P defined relative to the pre-operative bone images or 3-D virtual models for registration; trajectory parameters; the planned POSE of an implant in, on, or relative to a bone and/or a set of instructions to operate the surgical robot 202. The set of instructions may include instructions for the surgical robot to modify a volume of bone to receive an implant. The set of instructions may illustratively include: a cut-file having a set of cutting parameters/instructions (e.g., cut paths, velocities) to automatically modify the volume of bone; a set of virtual boundaries defined to haptically constrain a tool within the defined boundaries to modify the bone; a set of boundaries coupled with power or actuation control of a tracked surgical device to ensure the end-effector only removes bone within the boundaries; a set of planes or drill holes to drill pins or tunnels in the bone; implantation instructions for placing, orienting, or aligning an implant on, in, or relative to a bone; or a graphically navigated set of instructions for modifying the tissue. In particular inventive embodiments, the set of instructions includes a cut-file and implantation instructions for execution by a surgical robot to automatically modify the volume of bone and place the implant, which is advantageous from an accuracy and usability perspective. The surgical plan data generated from the planning computer 716 may be transferred to the device computer 718 and/or tracking computer 720 through a wired or wireless connection in the operating room (OR); or transferred via a non-transient data storage medium (e.g., a compact disc (CD), a portable universal serial bus (USB) drive) if the planning computer 716 is located outside the OR. It is appreciated that the planning computer functions may be spread over more than one device, and may be performed all or part in cloud computing environment.

The device computer 718 in some inventive embodiments is housed in the moveable base 708 and contains hardware, software, data and utilities that are preferably dedicated to the operation of the surgical robotic device 702. This may include surgical device control, robotic manipulator control, the processing of kinematic and inverse kinematic data, the execution of registration algorithms, the execution of calibration routines, the execution of the set of instructions (e.g., cut-files, implantation instructions, the trajectory parameters), coordinate transformation processing, providing workflow instructions to a user, and utilizing position and orientation (POSE) data from the tracking system 706. It is appreciated that the device computer functions may be spread over more than one device and may be performed all or part on the planning computer or in cloud computing environment. In some embodiments, the surgical system 700 includes a mechanical digitizer arm 705 attached to the base 708. The digitizer arm 705 may have its own tracking computer or may be directly connected with the device computer 718. The mechanical digitizer arm 705 may act as a digitizer probe that is assembled to a distal end of the mechanical digitizer arm 705. In other inventive embodiments, the system includes a tracked hand-held digitizer device 736 with a probe tip.

The tracking system 706 may be an optical tracking system that includes two or more optical receivers 707 (e.g., cameras) to detect the position of fiducial markers (e.g., retroreflective spheres, active light emitting diodes (LEDs)) uniquely arranged on rigid bodies or integrated directly with the tracked object. The arrangement of a plurality of fiducial markers is referred to herein as a tracking array (738a, 738b, 738c, 738d), where each tracking array has a unique arrangement of fiducial markers, or a unique transmitting wavelength/frequency if the markers are active LEDs. An example of an optical tracking system is described in U.S. Pat. No. 6,061,644. The tracking system 706 may be built into a surgical light, located on a boom, a stand 740, or built into the walls or ceilings of the OR. The tracking system computer 720 may include tracking hardware, software, data, and utilities to determine the POSE of objects (e.g., bones B, surgical device 702) in a local or global coordinate frame. The POSE of the objects is collectively referred to herein as POSE data, where this POSE data may be communicated to the device computer 718 through a wired or wireless connection. Alternatively, the device computer 718 may determine the POSE data using the position of the fiducial markers detected from the optical receivers 707 directly.

The POSE data is determined using the position data detected from the optical receivers 707 and operations/processes such as image processing, image filtering, triangulation algorithms, geometric relationship processing, registration algorithms, calibration algorithms, and coordinate transformation processing.

The POSE data is used by the computing system 704 during the procedure to update the POSE and/or coordinate transforms of the bone B, the surgical plan, and the surgical robot 702 as the manipulator arm 710 and/or bone(s) (A, F) move during the procedure, such that the surgical robot 702 may accurately execute the surgical plan.

In another inventive embodiment, the surgical system 700 does not include an optical tracking system 706, but instead employs a bone fixation and monitoring system to fix the bone directly to the surgical robot 202 and to monitor bone movement as described in U.S. Pat. No. 5,086,401.

The planning computer 716, device computer 718, and/or tracking computer 220 further includes one or more processors, and non-transient memory having software executable instructions stored therein for performing the embodiments described herein. For example, with reference to FIG. 10, the end-effector 711 may include an assembly device 742 (e.g., a gripping clamp, magnet) for assembling the end-effector 711 to the non-spherical femoral head implant 201 as originally described. The initial orientation of the femoral head implant 201 may be determined by registering the femoral head implant 201 when assembled to the end-effector 711, or by way of one or more reference holes or pins that receives the end-effector 711 to provide the initial position and/or orientation of the femoral head implant 201 relative to the end-effector 711. The computer-assisted surgical system 700 may then place and orient the femoral head implant 201 on the neck 103 in the planned orientation according to the pre-operative plan. It should be appreciated that the end-effector 711 may assemble with other implants or implant components (e.g., a modular neck component 103, cup implant (401, 410)) in this manner as originally described.

### OTHER EMBODIMENTS

The present invention also includes a business method in which one or more aspects of the method of pre-surgical planning, implant design, implant placement/positioning are performed for financial remuneration. The subject receiving an implant or a third party insurer is invoiced for such services. Payment is then conveyed by electronic transaction or financial instrument to the provider of the method for services rendered and the implant.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes may be made in the function and arrangement of elements without departing from the scope as set forth in the appended claims and the legal equivalents thereof.

1. A method for implantation of an implant comprising:
devising a pre-surgical plan with pre-operative planning software operating on a computer to define at least one of shape, orientation, type, size, geometry, or placement of the implant in, on, or relative to a bone of a subject; and
a computer-assisted surgical device positioning the implant in, on, or relative to the bone in accordance with the pre-surgical plan
the implant having at least a head component or a cup component, the head component or the cup component having at least two cross-sectional radii of different dimensions.

2. The method of claim 1 further comprising rotating the implant to an orientation defined in the pre-surgical plan.

3. The method of claim 1 further comprising assembling the implant to the computer-assisted surgical device and determining an initial position or orientation of the implant relative to the computer-assisted surgical device.

4. The method of claim 3 wherein the computer-assisted surgical device comprises a serial-chain manipulator system, a parallel robotic system, a haptically controlled robotic system, a hand-held robotic system, or navigated or tracked set of surgical instruments.

5. The method of claim 1 wherein the computer-assisted surgical device comprises a surgical robot having an end-effector.

6. The method of claim 5 further comprising assembling the implant to the end-effector, and determining an initial position or orientation of the implant relative to the surgical robot.

7. The method of claim 6 wherein the end-effector includes a gripper, a magnet, or a portion of the end-effector is received in one or more holes in the implant.

8. The method of claim 6 further comprising the end-effector rotating, translating, or a combination of rotating and translating the implant to a position or orientation as defined in the pre-surgical plan.

9. The method of claim 1 wherein the implant is replacing a ball and socket joint.

10. The method of claim 1 wherein the implant further comprises a second complementary component.

11. The method of claim 10 wherein the second complementary component comprises a stem component, a neck component, or a stem and neck component.

12. The method of claim 11 wherein the implant is only the head component.

13. The method of claim 1 wherein the head component or cup component is ellipsoidal.

14. The method of claim 1 further comprising preparing at least a portion of the bone for the placement of the implant by the computer-assisted surgical device.

15. The method of claim 1 further comprising rotating the implant to an anteversion as defined in the pre-surgical plan with the computer-assisted surgical device.

16. The method of claim 1 wherein the implant is configured for implantation in at least one of a knee joint, a hip joint, a spine, a shoulder joint, an elbow joint, an ankle joint, a jaw, a tumor site, a hand joint, or a foot joint.

17. A system for performing the method of claim 1 comprising:
a planning workstation for devising a pre-surgical plan;
a computer-assisted surgical device to adapted to engage and position an implant in, on, or relative to a bone in accordance with the pre-surgical plan; and
wherein the implant has at least a head component or a cup component, the head component or the cup component having at least two cross-sectional radii of different dimensions.

18. The system of claim 17 wherein the computer-assisted surgical device comprises a serial-chain manipulator system, a parallel robotic system, a haptically controlled robotic system, a hand-held robotic system, or navigated or tracked set of surgical instruments.

19. The system of claim 17 wherein the computer-assisted surgical device is a surgical robot.

20. The system of claim 19 wherein the surgical robot comprises an end-effector adapted to couple the implant with a bone or a second complementary component.
